(19) United States
(12) Patent Application Publication
(10) Pub. No.: US 2018/0260526 A1
(43) Pub. Date: Sep. 13, 2018

(54) COGNITIVE PAIN MANAGEMENT AND MAPPING ASSOCIATIONS

(71) Applicant: International Business Machines Corporation, Armonk, NY (US)

(72) Inventors: Nadiya Kochura, Bolton, MA (US); Fang Lu, Billerica, MA (US)

(21) Appl. No.: 15/452,901

(22) Filed: Mar. 8, 2017

(51) Int. Cl. G06F 19/00; A61B 5/00

(52) U.S. Cl. CPC G06F 19/322; A61B 5/4824

(57) ABSTRACT

Embodiments describing an approach to receiving a user's pain description, and aggregating, the user's data. Generating, a population group based on the user's data, displaying, processors, a plurality of suggested pain descriptions for selection. Responsive to receiving the user's selection for the plurality of suggested pain description selection, producing, a preliminary pain level based on user, and generating, by one or more processors, a weighted pain level.

### BACKGROUND OF THE INVENTION

The present invention relates generally to the field of cognitive pain mapping, and more particularly to cognitive pain mapping through management association.

Pain is an important reaction to external and/or internal discomforts and/or injuries by the human body. Currently, due to the increase in prescriptions of pain medication, the issue of proper pain diagnosis and pain management is crucially needed. The intensity of pain helps individuals and medical professionals distinguish the degree of discomfort and/or the extent of an injury. Pain intensity and the intensity of the injury and/or discomfort are typically directly correlated, however, pain intensity can be very subjective and vary greatly between individuals. The lack of a system and/or method of bridging the gap between subjective and objective pain is important to the advancement of pain management. There have been attempts to efficiently and effectively map patient pain levels whether it is through images, facial monitoring and/or recognition, bio-marker labeling and/or tracking, and various other means known in the art; however, the need for effectively and efficiently mapping subjective to objective pain levels still exists.

### SUMMARY

According to one embodiment of the present invention, a method, computer program product, and computer system for pain mapping and/or pain evaluation. A computer implemented method includes receiving, by one or more processors, a user's pain description. Aggregating, by one or more processors, a user's data. Generating, by one or more processors, a population group based on the user's data. Displaying, by one or more processors, a plurality of suggested pain descriptions for selection. Responsive to receiving the user's selection for the plurality of suggested pain description selection, producing, by one or more processors, a preliminary pain level based on user, and generating, by one or more processors, a weighted pain level.

### BRIEF DESCRIPTION OF THE DRAWINGS

FIG. 1 is a functional block diagram illustrating a distributed data processing environment, in accordance with an embodiment of the present invention;

FIG. 2 is a block diagram illustrating a data processing environment within pain mapping component, within the distributed data processing environment of FIG. 1, for generating pain levels, in accordance with an embodiment of the present invention;

FIG. 3 illustrates operational steps of a pain mapping component, on a mobile device within the distributed data processing environment of FIG. 1, in accordance with an embodiment of the present invention; and

FIG. 4 depicts a block diagram of components of the server computer executing the intelligent mapping program within the distributed data processing environment of FIG. 1, in accordance with an embodiment of the present invention.

### DETAILED DESCRIPTION

People often describe and/or associate pain and/or discomfort with past experiences where they felt similar pain and/or discomfort. However, these pain descriptions are very subjective and vary between individuals by depending on a person's personal pain tolerance. In some instances, people describe pain sensation by referring to previous pain events as a referential baseline. For example, if a person trips and falls while running, in order to describe the pain, they might state that their leg hurts as much as when the fell off of their bike. This description of pain doesn't really define any true measurement and/or level of pain and is merely a subjective description that the person is experiencing pain. In some instances, people can refer to the same pain trigger event while describing different levels of pain. The referential baseline(s) or association(s) between the pain level and prior accident(s) or pain trigger event(s) differ between various population groups. Therefore, creating a lot of uncertainty within the pain management community.

However, embodiments of the present invention improve the previously limited method of pain mapping and have the ability to generate a pain map combining subjective and objective pain descriptions. For example, a pain level and pain management regime can be generated through analysis of a user's pain description, pain trigger even, user medical history, and other data related to the user's pain description, pain trigger even, user medical history retrieved from a knowledge repository. In various embodiments of the present invention, a medical professional can track the progress of a user's pain and properly manage the users pain without over prescribing pain medication. Embodiments of the present invention recognize that there is a need to map pain descriptions provided by patients to some conventional pain scale system. Generating a pain map based off of pain descriptions could help reduce the issue of over medicating patients by improving pain level ratings/mapping. Cross referencing a patient's subjective pain descriptions against a pool of similar subjective and objective pain descriptions will provide medical professional with a baseline of what pain level patients are really enduring, and improve the are pain mapping and pain management.

Implementation of embodiments of the invention may take a variety of forms, and exemplary implementation details are discussed subsequently with reference to the Figures.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product. The computer program product may include a computer readable storage medium (or media) having computer readable program instructions thereon for causing a processor to carry out aspects of the present invention.

The computer readable storage medium can be any tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It can also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations can be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

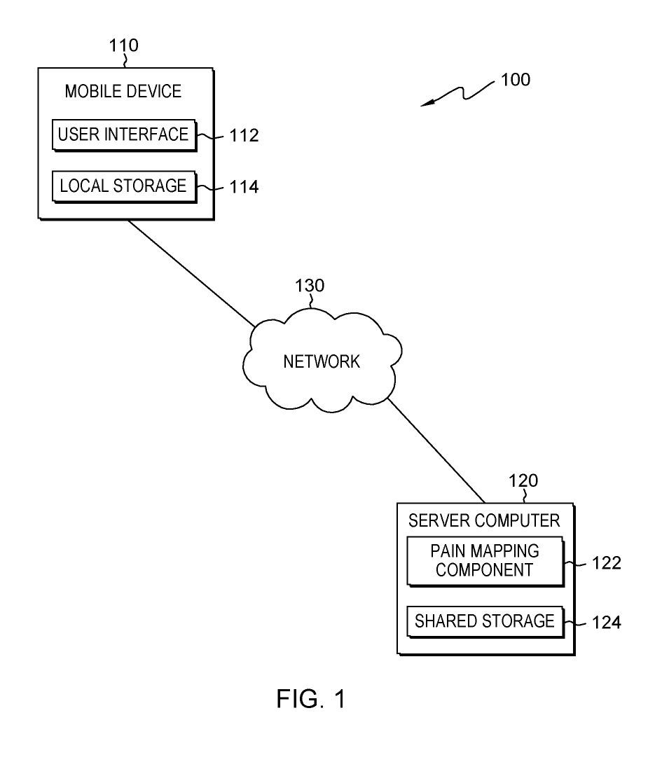

FIG. 1 is a functional block diagram illustrating a distributed data processing environment, generally designated 100, in accordance with one embodiment of the present invention. The term “distributed” as used in this specification describes a computer system that includes multiple, physically distinct devices that operate together as a single computer system. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

Distributed data processing environment 100 includes mobile device 110 and server computer 120 interconnected over network 130. Network 130 can be, for example, a telecommunications network, a local area network (LAN), a wide area network (WAN), such as the Internet, or a combination of the three, and can include wired, wireless, or fiber optic connections. Network 130 can include one or more wired and/or wireless networks that are capable of receiving and transmitting data, voice, and/or video signals, including multimedia signals that include voice, data, and video information. In general, network 130 can be any combination of connections and protocols that will support communications between mobile device 110 and/or server computer 120. In various embodiments, not depicted in FIG. 1, network 130 can be any combination of connections and protocols that will support communications between mobile device 110, server computer 120, and/or a separate third party mobile device.

In various embodiments, mobile device 110 can be, but is not limited to, a standalone device, a server, a laptop computer, a tablet computer, a netbook computer, a personal computer (PC), a smart phone, a desktop computer, a smart television, a smart watch, or any combination thereof. In general, mobile device 110 can be representative of any programmable mobile device and/or a combination of programmable mobile devices capable of executing machine-readable program instructions and communicating with users of other mobile devices via network 130 and/or capable of executing machine-readable program instructions and communicating with server computer 120. In various embodiments, mobile device 110 can be a computer and/or mobile device attached and/or connected to medical equipment, in which the computer receives user data from the medical equipment, generally computer that is able to receive user feedback and/or information while feeding and/or monitoring a user's vital signs. For example, a computer receiving a user's blood pressure, heart rate and/or oxygen level while a user describes their pain.

Local storage 114 and shared storage 124 are data repositories that may be written to and read by one or a combination of pain mapping component 122, user interface 112, server computer 120, and or all components and applications of mobile device 110 and server computer 120 known in the art. Local storage 114 and shared storage 124 can be connected via network 130 or connected through a cable and or wired connection. Local storage 114 and Shared storage 124 can be hard drives, memory cards, computer output to laser disc (cold storage), and or any form of data storage known in the art. In one embodiment, not illustrated in FIG. 1, local storage 114 can be within server computer 120 and accessed via network 130. In one embodiment, pain mapping component 122 can automatically access local storage 114 and/or shared storage 124, via network 130 and begin analyzing data. In various embodiments, pain mapping component 122 can access local storage and/or shared storage in order to create a pain map and generate an accurate pain level using objective and subjective data and/or information.

Mobile device 110 includes a user interface (UI) 112, which executes locally on mobile device 110 and operates to provide a UI to a user of mobile device 110. User interface 112 further operates to receive user input from a user via the provided user interface, thereby enabling the user to interact with mobile device 110. In one embodiment, user interface 112 provides a user interface that enables a user of mobile device 110 to interact with pain mapping component 122. In various embodiments, a user can edit pain mapping component 122 program settings, designated language and/or user settings, via a mobile application, website, integrated mobile settings, remote server, and any combination thereof. For example, pain mapping component 122 enables a medical professional and/or user to create and/or generate a personalized medical profile. In various embodiments, UI 112 can receive, display, and/or emit sound, brail, images, videos, pictures, and/or vibrations. In other embodiments, can receive voice command instructions.

Server computer 120 may be a desktop computer, a laptop computer, a tablet computer, a specialized computer server, a smartphone, server computer or any other computer system known in the art. In certain embodiments, server computer 120 represents a computer system utilizing a cluster computers and components that act as a single pool of seamless resources when accessed through network 130, as is common in data centers and with cloud computing applications. In general, server computer 120 is representative of any programmable mobile device or combination of programmable client devices capable of executing machine-readable program instructions and communicating with other computer devices via a network (i.e., network 130).

In various embodiments, pain mapping component 122 can generate a map between a user given pain description (i.e., subjective pain description) and a conventional pain scale system (i.e., objective pain description). Generally, pain mapping component 122 can generate a weighted pain level using a subjective description(s) and objective pain scale(s). For example, pain mapping component 122 can receive user pain descriptions and/or pain trigger events, and evaluate them against other similar user pain descriptions and/or pain trigger events. Additionally, in this particular example, pain mapping component 122 can integrate the gathered patient data and/or subjective data with a conventional pain scale system, in order to produce a weighted pain level. In other embodiments, pain mapping component 122 can integrate the gathered patient and/or subjective data and analyze it against a conventional pain scale system, in order to produce a weighted pain level. A user's pain description can be any description of pain by a user and/or patient. For example, a user and/or patient may describe their pain by stating “my throat hurts as much as when I swallowed hot tea.” A pain trigger event can be any event that caused the user the pain they are currently describing and/or caused the user pain in the past. In other embodiments, a pain trigger event can be retrieved from system knowledge repository (SKR) 202 and/or shared storage 124 by pain mapping component 122.

In other embodiments, pain mapping component 122 can generate a population group for a user. In various embodiments, pain mapping component 122 can generate a taxonomy for the population group. In various embodiments, pain mapping component 122 resides on server computer 120; however, in other embodiments, pain mapping component 122 can reside on mobile device 110, a server computer not depicted in environment 100, a mobile device not depicted in environment 100, network 130, and/or or a cloud based service provider. In various embodiments, pain mapping component 122 can receive audio, visual (e.g., images, graphs, figures and/or videos), text, and/or any other form of communication known in the art. In exemplary embodiments, pain mapping component 122 can use machine learning, neuro-linguistic programing, and/or any other form of cognitive learning known in the art to analyze and/or generate pain levels.

Generally, in various embodiments, pain mapping component 122 acquires the most common associations between pain levels and pain trigger accidents or events across various population groups and creates a knowledge database that maps the pain level, pain trigger event and their descriptions across carious population groups. In various embodiments, pain mapping component 122 applies the knowledge obtained and/or generated from SKR 202 to evaluate a user's level of pain and stages of recovery. In other embodiments, pain mapping component 122 can be connected and/or integrated with a super computer and/or artificial intelligence. In various other embodiments, pain mapping component 122 can be used by a medical professional to track a user's pain progress and pain management, in which pain mapping component 122 can assist the medical professional in proscribing the adequate amount of pain medication based on the generated weighted pain level, which encompasses cased study and/or data from similar pain descriptions. For example, a medical professional can use pain mapping component 122 to track the pain progress/pain management of a patient suffering from a broken arm.

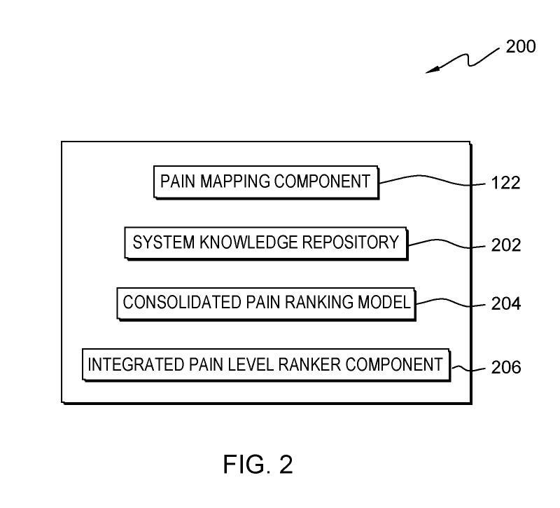

Continuing to illustrate this example, the medical professional can run pain mapping component 122 to consistently update the users pain level and track the user pain progress and/or healing progress. In this particular example, pain mapping component 122 can also suggested an amount of appropriate pain medication for the patient based off the user data, pain description, data analysis, generated weighted pain level, and/or the case study/general data related to the user data on SKR 202. In various embodiments, pain mapping component 122 can retrieve similar and/or data and/or pain management information from SKR 202. For example, a user age 16, weighing 135 pounds, broke their arm playing football, and it was their first broken arm, subsequent to pain mapping component 122 receiving this information, pain mapping component 122 can retrieve other cases and/or incidents similar to the brake event and/or the user's data. Continuing to illustrate this example, pain mapping component 122 could display pain levels of previous pain treatment and pain levels for user's ages 14-18, weighing 120-150 pounds, who broke their playing a physical sport (i.e., basketball, football, rugby, soccer, skateboarding, etc.). In various embodiments, a user can set the search parameters and/or ranges. Pain mapping component 122 is depicted and described in further detail with respect to FIG. 2.

FIG. 2 is a functional block diagram illustrating a computing environment of pain mapping component 122, generally designated 200, in accordance with an embodiment of the present invention. FIG. 2 provides only an illustration of one implementation and does not imply any limitations with regard to the environments in which different embodiments may be implemented. Many modifications to the depicted environment may be made by those skilled in the art without departing from the scope of the invention as recited by the claims. Computing environment 200 includes pain mapping component 122. Pain mapping component 122 comprises system knowledge repository (SKR) 202, consolidated pain ranking model (CPRM) 204, and integrated pain level ranker component (IPLRC) 206.

In various embodiments, system knowledge repository (SKR) 202, is a subcomponent of pain mapping component 122 housed within server computer 120; however, SKR 202 can be housed within mobile device 110, and/or a cloud based service not depicted in FIG. 1. In various embodiments, SKR 202 can be a standalone device. Generally, SKR 202 may be housed anywhere in environment 100, as long as it remains a subcomponent of pain mapping component 122. In various embodiments, SKR 202 can be a database and/or data repository, in which data is collected and used to create pain level models, evaluate pain levels, and/or generate pain maps and/or pain levels. The data collected and/or stored in SKR 202 can be, but are not limited to, personal medical history and/or medical records, statistical data records from medical archives, general medical knowledge and/or history, previous pain evaluations, previous and/or present pain maps, previous and/or present population group taxonomy, pain trigger event descriptions, and/or subjective pain descriptions. In various embodiments, SKR 202 can tag and pull data relative to a user's pain description, pain trigger event, and/pain evaluation. For example, if a user fell off their bike and describes their pain similar to that when they fell out of a tree, then, SKR 202 would pull the users medical history, similar injury/pain description, similar pain trigger event, and/or population group. Generally, in various embodiments, SKR 202 can collect and/or analyze patient data, pain description, and/or pain trigger event.

Additionally, in this exemplary embodiment, SKR 202 can analyze the data against a database to generate a population group, a user profile, and/or a pain level. In other embodiments, SKR 202 is a knowledge repository that stores and collects data that can be accessed by pain mapping component 122, in which pain mapping component 122 can generate a pain map, pain level, and/or population group based on the aforementioned information acquired by SKR 202. In various embodiments, SKR 202 aggregates the user's data. In various embodiments, the aggregation and/or accumulation of data and/or user data can be labeled as a learning phase and/or data collection stage for SKR 202 and/or pain mapping component 142. For example, as the user discloses their pain description and/or user data to pain mapping component 142 SKR 202 learns about the user and molds the pain model to the user's needs based off of their data and/or previous user data.

In various embodiments, SKR 202 can be generated from a data collection stage. For example, a user and/or medical practitioner enters a user's pain description and/or pain trigger event into pain mapping component 122. In this particular example, once the data is entered into pain mapping component 122, SKR 202 is generated based on the data and/or information entered about the user and/or the user's pain description, pain level, and/or pain trigger event. In various embodiments, SKR 202 acquires knowledge about the most common associations between pain levels and pain trigger accidents or events across various population groups. In exemplary embodiments, SKR 202 maps the pain level(s) and/or pain trigger event(s) and their descriptions across various population groups. For example, SKR 202 can map the pain level and/or pain trigger event across a range of populations groups based on, but not limited to, age, gender, heritage, language, medical history, geographical region, nationality, general medical knowledge/information, previous pain maps, or any combination therein. SKR 202 can support a series of datasets comprising, but not limited to, pain trigger event descriptions linked to population group, patient pain descriptions, mapping between pain description and conventional standard pain scale 1-10, images, audio and data related to the pain trigger event, and/or attributes of the defined population groups and/or user profiles. In other embodiments, SKR 202 is a knowledge repository that stores and collects data that can be accessed by pain mapping component 122, in which pain mapping component 122 can generate a pain map, pain level, and/or population group based on the aforementioned information acquired by SKR 202. In various embodiments, SKR 202 aggregates the user's data.

In exemplary embodiments, SKR 202 can create and/or generate a population group, using the user's pain description, personal info, pain trigger event, and/or medical records/medical history. Associating the user with a certain population group enables pain mapping component 122 to produce a more accurate and relevant pain level assessment. In various embodiments, population groups comprise, but are not limited to, age, gender, heritage, language, social cues, demographics, occupation, medical history, pain thresholds, geographical region, visual association, psychological level, nationality, general medical knowledge/information, previous pain maps, or any combination therein. For example, someone who is allergic to bees is stung by a bee. In this particular example, this particular person would describe a strong/high pain level; however, a farmer or bee keeper would associate the bee sting to minor pain. In other embodiments, each population group can have specific pain trigger event descriptions. For example, adults versus children, a child when asked to describe the pain might state “it hurts as much as when I fell from the seesaw.” Versus, an adult who might physically describe the pain in more detail stating “the pain is a sharp radiating pain that feels like needles are pricking me.” In other embodiments, specific language terms, and/or specific pain trigger events can contribute to population group characteristics. Furthermore, in some embodiments, the repository scheme allows the patient to be a member of many population groups.

In various embodiments, consolidated pain ranking model (CPRM) 204, is a subcomponent of pain mapping component 122 housed within server computer 120; however, CPRM 204 can be housed within mobile device 110, and/or a cloud based service not depicted in FIG. 1. In various embodiments, CPRM 204 can be a standalone device. Generally, CPRM 204 may be housed anywhere in environment 100, as long as it remains a subcomponent of pain mapping component 122. Generally, in various embodiments, CPRM 204 can consolidate the data gathered and/or generated from SKR 202. In various embodiments, CPRM 204 comprises a pain level classifier. In various embodiments, pain mapping component 122 uses programming language to build the pain level classifier based on the user's pain description. In exemplary embodiments, pain mapping component 122 can use previously submitted user pain descriptions and pain scales; additionally, pain mapping component 122 can produce natural language statistical models during the data collection stage and/or learning stage. In other embodiments, pain mapping component 122 can use the population group label (ID) as an additional feature in model training.

In various embodiments, CPRM 204 can work in junction with IPLRC 206 to produce a pain level. In various embodiments, CPRM 204 can run the pain classifier to predict the users pain level based on user's pain description, analyze the user's medical history data and previous pain related records, conditions, and/or events, virtually assign the user to a predefined population group(s), establish that there are multiple descriptions of the similar pain trigger event and/or accidents stored in SKR 202, retrieve related pain descriptions found and present them to the user, request the user identify the pain description that most closely identifies with the user's experience and/or description, and analyze and/or determine whether the selected description is associated with the pain level originally reported by the user. Subsequent, to CPRM 204 conducting the aforementioned steps, in various embodiments, CPRM 204 can communicate the information it has gathered and distribute the information to IPLRC 206 so, IPLRC 206 can generate a pain level. In other embodiments, pain mapping component 122 can direct CPRM 204 to communicate and/or work with IPLRC 206.

In various embodiments, pain mapping component 122, can collect the following data for CPRM 204: level of pain predicted by the pain classifier using the pain language model, the level of pain on conventional scale estimated by the user, the level of pain that is mapped based on the users pain description and the users assigned population group(s), the level of pain that is mapped form the users pain description and the general population group, deviations in the pain users pain thresholds from the general population collected form the users data, deviations in the user's pain threshold and the relevant population group, the level of pain that is mapped to the most similar description selected by the patient from the presented options by CPRM 204 and stored in SKR 202, and/or the levels of pain associated with the pain trigger event(s). In other embodiments, CPRM 204 can collect the aforementioned data on its own and/or can be instructed by pain mapping component 122 to collect the aforementioned data.

In various embodiments, Integrated pain level ranker component (IPLRC) 206, is a subcomponent of pain mapping component 122 housed within server computer 120; however, IPLRC 206 can be housed within mobile device 110, and/or a cloud based service not depicted in FIG. 1. In various embodiments, IPLRC 206 can be a standalone device. Generally, IPLRC 206 may be housed anywhere in environment 100, as long as it remains a subcomponent of pain mapping component 122. In various embodiments, IPLRC 206 can learn and/or be trained to assign various weights to the pain model features. Generally, IPLRC 206 generates a pain level for the user based on the information and/or data from CPRM 204. In other embodiments, IPLRC 206 stores the user's data, the data collected from CPRM 204, and/or the generated pain level. The storing of the data contributes to the learning and/or training of pain mapping component 122 and/or IPLRC 206. The more IPLRC 206 and/or pain mapping component 122 are used and store information the smarter and more knowledgeable IPLRC 206 and/or pain mapping component 122 will become.

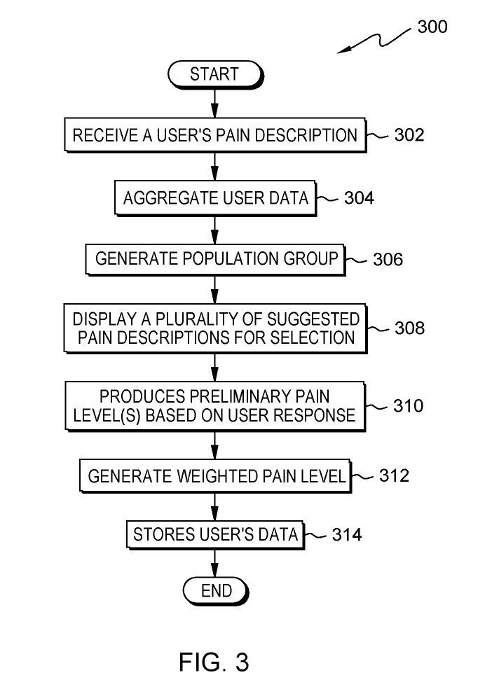

FIG. 3 is a flowchart depiction operational steps of pain mapping component 122, generally designated 300, on server computer 120 within distributed data processing environment 100 of FIG. 1, pain mapping and/or pain level evaluation, in accordance with an embodiment of the present invention. FIG. 3 provides only an illustration of one implementation and does not imply any limitations with regard to the environments in which different embodiments may be implemented. Many modifications to the depicted environment may be made by those skilled in the art without departing from the scope of the invention as recited by the claims.

In step 302, pain mapping component 122 receives a user's pain description and/or pain level. In various embodiments, pain mapping component 122 can prompt the user to describe their pain. For example, a user complaining about a sore throat would be prompted by pain mapping component 122, via user interface 112 (i.e., mobile device 110) to describe the users pain and enter a pain level/characterization of the pain. Continuing to illustrate this example, the user would then enter and/or describe their pain description stating “the pain feels as bad as when I was stung in the neck by a bee” and characterized the pain as a 5 (i.e., strong) on a scale from 1-10. In other embodiments, the user can be describing their pain to a medical professional who in turn would actually be entering the information into pain mapping component 122. In various embodiments, the user and/or medical professional submits the users pain description to pain mapping component 122, via mobile device 110, in which the keypad, camera, and/or microphone on user interface 112 are utilized to receive the user's pain description. In various embodiments, pain mapping component 122 initiates a learning phase, in which SKR 202 receives user data and/or information, user pain description, and/or retrieves data related to the user pain description and/or user data.

In step 304, pain mapping component 122 aggregates user data. In various embodiments, SKR 202 collects the user's data and/or opens the users profile. For example, subsequent to pain mapping component 122 receiving a user's pain description and/or characterization, SKR 202 accesses the users profile and/or user data. In various embodiment, the users profile and/or user data can be, but not limited to, age, gender, heritage, language, medical history, geographical region, nationality, general medical knowledge/information, previous pain maps, or any combination therein. The aggregation of user data enables pain mapping component 122 to effectively and accurately map subjective and objective pain descriptions, and/or generate effectively and accurately generate population groups (Step 306). In various embodiments, SKR 202 can create a user profile if a user does not possess a preexisting profile.

In step 306, pain mapping component 122 generates a population group. In various embodiments, data is pulled from SKR 202 to generate a population group based on the user's pain description, user data and/or pain level. In various embodiments, the data pulled form SKR 202 can be user data. In various embodiments, pain mapping component 122 tags keywords in the user's pain description and/or pain level, in which the tags are used to pull related data from SKR 202 to generate the population group based on the data, user data, and tagged keywords from the user's pain description. For example, a population group can comprise, but is not limited to, age, gender, heritage, language, social cues, demographics, occupation, medical history, pain thresholds, geographical region, visual association, psychological level, nationality, general medical knowledge/information, previous pain maps, or any combination therein. Continuing the example from step 304, the user is allergic to bees and is in the senior demographic age group.

In this particular example, SKR 202 would generate a population group containing the user's medical records, past pain description accounts referencing bee stings and/or allergic to bee stings and sore throats, previous accounts referencing seniors, bee stings, pain level/characterization 5 and sore throats, and previously generated pain levels from bee stings and/or sore throats in seniors. In various embodiments, pain mapping component 122 pulls data from SKR 202 to generate the population group. In other embodiments SKR 202 generates the population group. In various embodiments, subsequent to the learning phase, pain mapping component 122 can initiate a run phase, in which encompasses step 306 through step 314. For example, after pain mapping component 122 learns about the user pain description and/or pain event pain mapping component 122 will begin a run phase analyzing the user pain description and/or pain even and generate a pain map resulting in a weighted pain level.

In step 308, pain mapping component 122 displays suggested pain descriptions for selection. In various embodiments, subsequent to generating the population group CPRM 204 analyzes the generated population group, the user's medical history data, the user's previous pain related records, the user's previous conditions, and/or current and/or previous pain events, and virtually assign the user to a predefined population group(s). Further illustrating this particular various embodiments, establish that there are multiple descriptions of the similar pain trigger event and/or accidents stored in SKR 202, retrieve related pain descriptions found and present them to the user. In various embodiments, CPRM 204 will pull a selection of pain description scenarios that are similar to the user's pain description and ask the user to select the pain description scenario that relates best to their pain description. For example, continuing the example in step 306, CPRM 204 retrieves pain descriptions from SKR 202 that match the user's symptoms and mapped to pain level 5. In this particular example, CPRM 204 displays (a) “it hurts as if I swallowed a very hot beverage,” (b) “it hurts as if my throat has been poked by sharp needles,” and (c) “it feels like a fish bone is stuck somewhere in my throat and every time I swallow it hurts.” Continuing to illustrate this particular example, the user can than select the option they feel is the closest association to their pain description, via user interface 112.

In step 310, pain mapping component 122 produces preliminary pain level(s) based on user response. In various embodiments, a user can selection displayed pain description that best fits their situation and CPRM 204 can generate preliminary pain levels. For example, continuing the example in step 308, the user selects the displayed option (a) “it hurts as if I swallowed a very hot beverage.” In this particular example, subsequent to the user selection option (a), CPRM 204 detects that most patients with similar symptoms and an allergy to bee stings mapped the description (a) to a pain level of 7 and a pain level of 5 for the general population. Furthermore, in this particular example, CPRM 204 detects that the event of bee sting is mapped to a pain level of 6 for the user's population group. In various embodiments, the preliminary pain level(s) produced/generated by pain mapping component 122 can be responsive to receiving the user's selection for the plurality of suggested pain description selection. Generally, in various embodiment, the preliminary pain level generated by pain mapping component 122 can be responsive and/or determined by the selected pain description options displayed.

In step 312, pain mapping component 122 generates a weighted pain level. In various embodiments a weighted pain level can be the final pain level. In various embodiments, IPLRC 206 compares and analyzes the pain descriptions population group, and preliminary pain level(s) to generate the weighted pain level. For example, continuing the example in step 310, IPLRC 206 will analyze the data from SKR 202 and CPRM 204 and determine the weighted pain level to be 9 out of a scale from (1-10). In other embodiments, IPLRC 206 can recommend treatment for the user to medical professionals based off the weighted pain level.

In step 314, pain mapping component 122 stores and records the collected data. In various embodiments, IPLRC 206 can store and record the collected user's data, population groups, preliminary pain levels and/or weighted pain level. For example, subsequent to IPLRC 206 generating a weighted pain level, IPLRC 206 can store/save the user's data to SKR 202 and/or shared storage 124, and record the data to a user's medical chart, medical history file, and/or user profile. In various embodiments, if the user doesn't have a profile IPLRC 206 can make one for the user, but if the user already has a preexisting profile IPLRC 206 can update the profile with the new data.

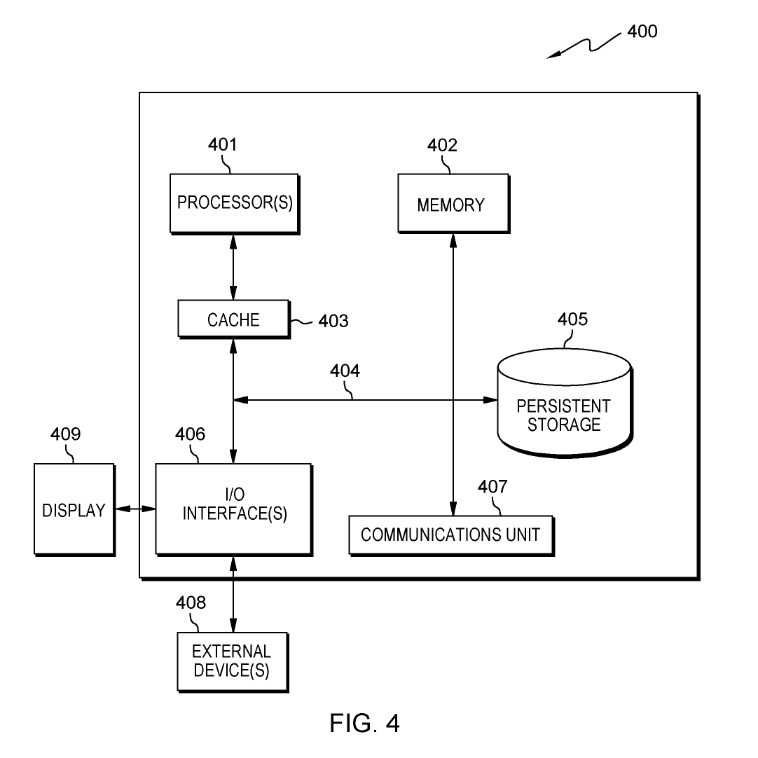

FIG. 4 depicts a block diagram of components of server computer 120 within distributed data processing environment 100 of FIG. 1, in accordance with an embodiment of the present invention. It should be appreciated that FIG. 4 provides only an illustration of one implementation and does not imply any limitations with regard to the environments in which different embodiments can be implemented. Many modifications to the depicted environment can be made.

FIG. 4 depicts a block diagram of components of a computing device within distributed data processing environment 100 of FIG. 1, in accordance with an embodiment of the present invention. It should be appreciated that FIG. 3 provides only an illustration of one implementation and does not imply any limitations with regard to the environments in which different embodiments can be implemented. Many modifications to the depicted environment can be made.

FIG. 4 depicts computer system 400, where server computer 120 represents an example of computer system 400 that includes pain mapping component 142. The computer system includes processors 401, cache 403, memory 402, persistent storage 405, communications unit 407, input/output (I/O) interface(s) 406 and communications fabric 404. Communications fabric 404 provides communications between cache 403, memory 402, persistent storage 405, communications unit 407, and input/output (I/O) interface(s) 406. Communications fabric 404 can be implemented with any architecture designed for passing data and/or control information between processors (such as microprocessors, communications and network processors, etc.), system memory, peripheral devices, and any other hardware components within a system. For example, communications fabric 404 can be implemented with one or more buses or a crossbar switch.

Memory 402 and persistent storage 405 are computer readable storage media. In this embodiment, memory 402 includes random access memory (RAM). In general, memory 402 can include any suitable volatile or non-volatile computer readable storage media. Cache 403 is a fast memory that enhances the performance of processors 401 by holding recently accessed data, and data near recently accessed data, from memory 402.

Program instructions and data used to practice embodiments of the present invention may be stored in persistent storage 405 and in memory 402 for execution by one or more of the respective processors 401 via cache 403. In an embodiment, persistent storage 405 includes a magnetic hard disk drive. Alternatively, or in addition to a magnetic hard disk drive, persistent storage 405 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 405 may also be removable. For example, a removable hard drive may be used for persistent storage 405. Other examples include optical and magnetic disks, thumb drives, and smart cards that are inserted into a drive for transfer onto another computer readable storage medium that is also part of persistent storage 405.

Communications unit 407, in these examples, provides for communications with other data processing systems or devices. In these examples, communications unit 407 includes one or more network interface cards. Communications unit 407 may provide communications through the use of either or both physical and wireless communications links. Program instructions and data used to practice embodiments of the present invention may be downloaded to persistent storage 405 through communications unit 407.

I/O interface(s) 406 enables for input and output of data with other devices that may be connected to each computer system. For example, I/O interface 406 may provide a connection to external devices 408 such as a keyboard, keypad, a touch screen, and/or some other suitable input device. External devices 408 can also include portable computer readable storage media such as, for example, thumb drives, portable optical or magnetic disks, and memory cards. Software and data used to practice embodiments of the present invention can be stored on such portable computer readable storage media and can be loaded onto persistent storage 405 via I/O interface(s) 406. I/O interface(s) 406 also connect to display 409.

Display 409 provides a mechanism to display data to a user and may be, for example, a computer monitor.

What is claimed is:

1. A method for improving pain mapping in patients, the method comprising:
receiving, by one or more processors, a user's pain description;
aggregating, by the one or more processors, a user's data
creating, by the one or more processors, a user profile based on the user's data
generating, by the one or more processors, a population group based on the user's data;
displaying, by the one or more processors, a plurality of suggested pain descriptions for selection;
responsive to receiving the user's selection to the plurality of suggested pain description selection, producing, by the one or more processors, a preliminary pain level based on the user's selection of the suggested pain descriptions;
analyzing, by the one or more processors, the user's data with a conventional pain scale system, the population group, and the preliminary pain level;
generating, by the one or more processors, a weighted pain level based on the analysis of the user's data with a conventional pain scale system, the population group, and the preliminary pain level; and
tracking, by one or more processors, the user's pain progress and pain management based on the weighted pain level, wherein the tracking is used to prescribe pain medication based on the generated weighted pain level.

2. The method of claim 1, further comprising:
storing, by the one or more processors, the user's data.

3. The method of claim 2, further comprising:
recording, by the one or more processors, the user's data to a user profile.

4. (canceled)

5. (canceled)

6. The method of claim 1, wherein, user data comprises: age, gender, heritage, language, social cues, demographics, occupation, medical history, pain thresholds, geographical region, visual association, psychological level, nationality, general medical knowledge/information, previous pain maps, or any combination therein.

7. The method of claim 1, wherein, the population group comprises: age, gender, heritage, language, social cues, demographics, occupation, medical history, pain thresholds, geographical region, visual association, psychological level, nationality, general medical knowledge/information, previous pain maps, or any combination therein.

8. A computer program product for improving pain mapping in patients, the computer program product comprising:
one or more computer readable storage devices and program instructions stored on the one or more computer readable storage devices, the stored program instructions comprising:
program instructions to, receive a user's pain description;
program instructions to, aggregate a user's data;
program instructions to create a user profile based on the user's data;
program instructions to, generate a population group based on the user's data;
program instructions to, display a plurality of suggested pain descriptions for selection;
responsive to receiving the user's selection to the plurality of suggested pain description selection, program instruction to, produce a preliminary pain level based on the user's selection of the suggested pain descriptions;
program instructions to analyze the user's data with a conventional pain scale system, the population group, and the preliminary pain level;
program instructions to, generate a weighted pain level based on the analysis of the user's data with a conventional pain scale system, the population group, and the preliminary pain level; and
tracking, by one or more processors, the user's pain progress and pain management based on the weighted pain level, wherein the tracking is used to prescribe pain medication based on the generated weighted pain level.

9. The computer program product of claim 8, further comprising:
program instructions to, store the user's data.

10. The computer program product of claim 9, further comprising:
program instructions to, record the user's data to a user's profile.

11. (canceled)

12. (canceled)

13. The computer program product of claim 8, wherein, user data comprises: age, gender, heritage, language, social cues, demographics, occupation, medical history, pain thresholds, geographical region, visual association, psychological level, nationality, general medical knowledge/information, previous pain maps, or any combination therein.

14. The computer program product of claim 8, wherein, the population group comprises: age, gender, heritage, language, social cues, demographics, occupation, medical history, pain thresholds, geographical region, visual association, psychological level, nationality, general medical knowledge/information, previous pain maps, or any combination therein.

15. A computer system for improving pain mapping in patients comprising:
one or more computer processors;
one or more computer readable storage devices;
program instructions stored on the one or more computer readable storage devices for execution by at least one of the one or more computer processors, the stored program instructions comprising:
program instructions to, receive a user's pain description;
program instructions to, aggregate a user's data;
program instructions to create a user profile based on the user's data;
program instructions to, generate a population group based on the user's data;
program instructions to, display a plurality of suggested pain descriptions for selection;
responsive to receiving the user's selection to the plurality of suggested pain description selection, program instruction to, produce a preliminary pain level based on the user's selection of the suggested pain descriptions;
program instructions to analyze the user's data with a conventional pain scale system, the population group, and the preliminary pain level;
program instructions to, generate a weighted pain level based on the analysis of the user's data with a conventional pain scale system, the population group, and the preliminary pain level; and
tracking, by one or more processors, the user's pain progress and pain management based on the weighted pain level, wherein the tracking is used to prescribe pain medication based on the generated weighted pain level.

16. The computer system of claim 15, further comprising:
program instructions to, store the user's data.

17. The computer system of claim 16, further comprising:
program instructions to, record the user's data to a user's profile.

18. (canceled)

19. The computer system of claim 15, wherein, user data comprises: age, gender, heritage, language, social cues, demographics, occupation, medical history, pain thresholds, geographical region, visual association, psychological level, nationality, general medical knowledge/information, previous pain maps, or any combination therein.

20. The computer system of claim 15, wherein, the population group comprises: age, gender, heritage, language, social cues, demographics, occupation, medical history, pain thresholds, geographical region, visual association, psychological level, nationality, general medical knowledge/information, previous pain maps, or any combination therein.
